(19) United States
(12) Patent Application Publication
(10) Pub. No.: US 2010/0211080 A1
(43) Pub. Date: Aug. 19, 2010

(54) UMBILIGUARD

(76) Inventors: Dean Trivisani, Oakland, NJ (US); Pierro Galluzzo, Marlboro, NJ (US)

Correspondence Address: Gearhart Law LLC, 4 Ferndale Road, Chatham, NJ, 07928, US

(21) Appl. No.: 12/658,622

(22) Filed: Feb. 11, 2010

Related U.S. Application Data
(60) Provisional application No. 61/207,540, filed on Feb. 13, 2009.
(60) Provisional application No. 61/281,096, filed on Nov. 12, 2009.

(51) Int. Cl. A61B 17/12

(52) U.S. Cl. 606/120

(57) ABSTRACT

The present invention relates to an infant supervision device featuring an umbilical cord clamp having a hinge, jaws and a locking mechanism. The present invention also contains a wire or a strip of conducting material that runs through the jaws and the hinge and that forms a current carrying circuit as soon as terminals within the locking mechanism are linked, which occurs when the clamp is closed. The clamp has a first end with a hinge and a second end having a locking mechanism. The locking mechanism contains a sensor, which may be an active or passive transmitter, a receiver or a magnet. The sensor is capable of communicating with an external detection device, and alert child care monitors in an event that the clamp is broken or cut, or if an infant wearing the present invention is carried beyond the secure perimeter covered by the external detection device. The clamp may also be able to send out positioning and health related information to the external detection device by using micro waves, radio waive, ultra-sound waives or by communicating with a wireless network.

### CLAIM OF PRIORITY

This application claims priority to U.S. Provisional Application 61/207,540 filed Feb. 13, 2009, U.S. Provisional Application 61/281,096 filed Nov. 12, 2009 and US Design Application 29/354,639 filed Jan. 27, 2010, the contents of which are fully incorporated herein by reference.

### FIELD OF THE INVENTION

The invention relates to an infant supervision device, in particular, to an infant theft prevention device.

### BACKGROUND OF THE INVENTION

The invention relates to a surveillance system preferably for tracking and preventing theft of infants and newborns at neonatal facilities. Infant abduction from hospitals remains an ever present threat, forcing infant care facilities to implement elaborate anti-theft safeguards that range from staffing additional nursing and security personal to installing sophisticated surveillance equipment, such as electronic tags, motion sensors and closed circuit cameras. Additionally, most locations have implemented a form of label matching technique, where all authorized individuals, including the parents, possess a randomly generated number, typically on their wrist band, which matches the random number on an infant's ankle or wrist band. All of the techniques mentioned above are well known in the art.

However, despite the much improved state of theft detection and prevention, abductions still occur. One of the weaknesses of the presently available systems is that they are highly noticeable and readily perceptible as security devices. A thief's best strategy may be to alertly and quickly be able to perceive an apparent anti-theft system or procedure. Advance notice or awareness may give a thief the time and opportunity to plan or implement a circumventive technique.

The present invention attempts to address this problem by taking a device that is presently being used for the medicinal needs of an infant, and enhancing it by adding integrated and discrete security features that are not readily perceived, and once perceived are difficult to circumvent. Specifically, the present invention accomplishes this by adding security features to the umbilical cord clamp of a newborn., which will not be removed for any routine treatment by hospital personal since it is needed to prevent bleeding and infections of the navel and abdominal area of an infant. Therefore, its unauthorized or unexpected removal will immediately raise an alarm with the nursing and security staffs. For this reason also, a thief, who likely desires to keep an infant alive and healthy, will be reluctant to remove the present invention, or will replace it with an umbilical cord clamp of his or her own. In any event, once the present invention is attached to the umbilical cord or its stump, it cannot be removed without cutting or otherwise breaching the claimed device. Such tampering will set off an external sensor, which may also be capable of locating the breached device. The external sensor will also be able to detect whether someone is trying to remove an infant from the care facility while the infant is still wearing the claimed device.

### DESCRIPTION OF THE RELATED ART

U.S. Pat. No. 4,899,134 discloses a detectable element inconspicuously disposed in an umbilical cord clamp used to clamp the umbilical cord a newborn child allows the detection of unauthorized movement of the newborn child and prevents the possible abduction of the newborn child.

U.S. Pat. No. 5,006,830 teaches a method and device for deterring unauthorized removal of a newborn from a defined area has a locking umbilical clamp with an attached identification mark and an attached triggering device capable of triggering a detection system upon removal of the umbilical clamp from the defined area and a wristband with an identification mark thereon corresponding to the identification mark on the umbilical clamp for attachment to the wrist of a person authorized to remove the newborn from the defined area.

U.S. Pat. No. 6,144,303 concerns a tag for monitoring the security of a patient, the tag comprising, a housing having a wall, the wall having an inner surface and an outer surface, an electronic circuit located in the housing, the electronic circuit including an alarm circuit, including a capacitance measuring circuit, the capacitance measuring circuit having first and second electrodes, the first and second electrodes located adjacent the inner surface and in spaced relation from one another to form a capacitor, the alarm circuit having means for generating an alarm signal upon the capacitance measuring circuit detecting a level of capacitance corresponding to an alarm condition, whereby the outer surface of the housing is placed in contact with the patient, with the first and second electrodes capacitively coupled to the patient, but without the first and second electrodes in physical contact with the patient, the capacitance measuring circuit detects an alarm condition when the patient is no longer in contact with the outer surface of the tag.

U.S. Pat. No. 6,753,781 discloses, in one aspect of the invention, a dual-mode infrared/radio frequency (IR/RF) transmitter is secured within a wristband worn by the mother and within an ankle and/or wristband worn by the infant. In a matching mode of operation, IR signals are received by infrared receivers located within the various rooms of the hospital to precisely and automatically determine by proximity that mother and infant arc correctly united. In a presence detecting mode, RF signals from the infant's badge are detected by RF receivers located throughout the maternity ward of the hospital or throughout the hospital generally. It a security mode, RF receivers located proximate exits of either of the maternity ward and/or the hospital detect RF signals from the ankle and provide a signal to generate an alarm.

Various implements are known in the art, but fail to address all of the problems solved by the invention described herein. One embodiment of this invention is illustrated in the accompanying drawings and will be described in more detail herein below.

### SUMMARY OF THE INVENTION

The present invention relates to an infant supervision device featuring an umbilical cord clamp having a hinge, jaws and a locking mechanism. The present invention also contains a wire or a strip of conducting material that runs through the jaws and the hinge and that forms a current carrying circuit as soon as terminals within the locking mechanism are linked, which occurs when the clamp is closed. The clamp has a first end with a hinge and a second end having a permanently locking mechanism. The first and second jaws rotate around the hinge. The permanently locking mechanism contains a sensor, which may be, but is not limited to, an active or passive transmitter, a receiver or a magnet. The sensor is capable of communicating with an external detection device, and alert child care monitors in an event that the clamp is broken or cut, or if an infant wearing the present invention is carried beyond the secure perimeter covered by the external detection device. The clamp may also be able to send out positioning and health related information to the external detection device by using micro waves, radio waive, ultra-sound waives or by communicating with a wireless network. A power source powers the sensor and the circuit.

It is an object of the present invention to provide an infant theft prevention and detection device.

It is another object of the present invention to provide a security device that is simple and inexpensive to create and implement.

Yet another object of the present invention is to provide a security device that is built around a conventional umbilical cord clamp.

Still another object of the present invention is to provide a device having a sensor that is able to communicate with an external detection device.

Still another object of the present invention is to provide a device that is not readily recognizable as a surveillance device.

Yet another object of the present invention is to provide a supervision device having a locking mechanism that cannot be unlocked.

It is yet another object of the present invention to provide an infant supervision device that is able to alert and initiate an alarm response if the clamp is cut or carried away from a monitored area.

Yet another object of the present invention is to provide an infant supervision device capable of communicating its location to the external detection device.

Still another object of the present invention is to provide an infant supervision device that is easy to manufacture and integrate with existing medical and surveillance technology.

### BRIEF DESCRIPTION OF THE DRAWINGS

FIG. 1 is a side view of the preferred embodiment of the present invention, showing an open clamp.

FIG. 2 is a side view of the preferred embodiment of the present invention, showing a closed clamp.

FIG. 3 is a cross sectional side view of an alternative embodiment of the present invention.

FIG. 4 is a side view of an alternative embodiment of the present invention.

FIG. 5 is a top view of an alternative embodiment of the present invention.

FIG. 6 is a perspective view of an alternative embodiment of the present invention, shown as a sleeve to be placed over a conventionally used medical device.

FIG. 7 is a schematic representation of the internal circuitry of the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

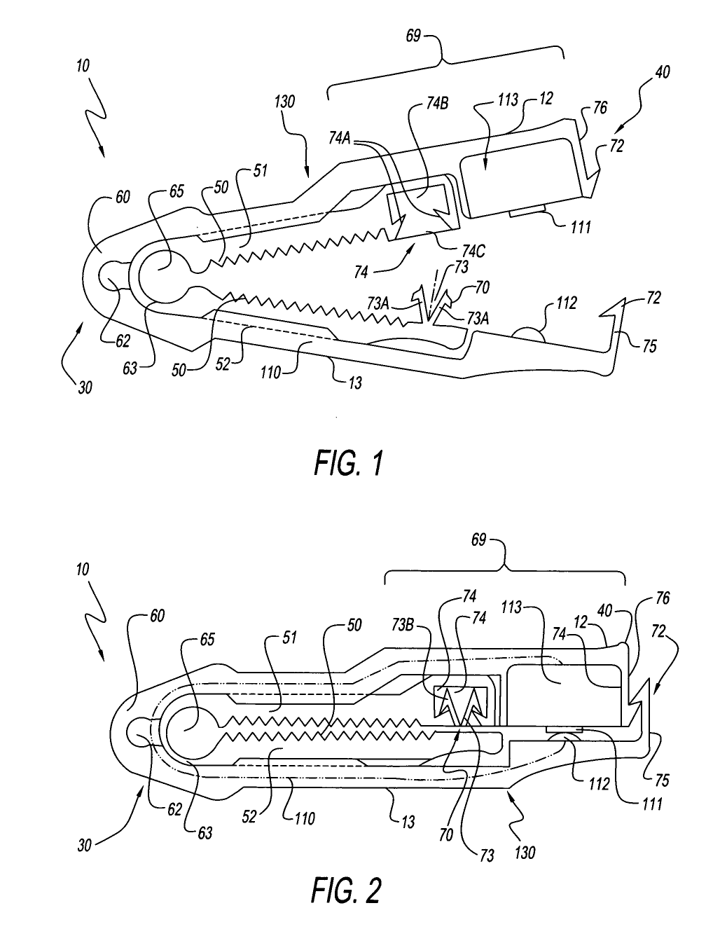

FIG. 1 shows a clamp 10, clamp top 12, clamp bottom 13, a first end 30, a second end 40, serrated edge 50, a first jaw 51, a second jaw 52, a hinge 60, a dimple opening 62, an inner hinge 63, an opening 65, a locking mechanism 69, a first locking mechanism 70, an additional locking mechanism 72, a clasp 73, V shaped hooks 73b, a latch 74, inwardly facing hooks 74a, a gap 74b, a recessed opening 74c, a hook 75, a catch 76, a wire 110, a first terminal 111, a second terminal 112, sensor 113, and a sleeve 130.

The clamp 10 functions as a conventional umbilical cord clamp and as a security device. The clamp 10 may be manufactured with the jaws 51 and 52 as an integral device or they may be fused together during manufacturing. The serrated edge 50 is placed around the umbilical cord of a newborn infant just before the umbilical cord is cut off from the placenta and is used to stop the bleeding and prevent infection in the remaining stump. The opening 65 is optional and is present as a backward compatibility feature, to accommodate the presently available wire or plug sensors (not shown). The opening 65 may be used to store the present invention on a ring threaded through the opening 65.

The hinge 60 and the inner hinge 63 are shown in an embodiment where the present invention is made as a sleeve 130 over a conventionally available clamp. Alternatively, only a single hinge 60 may be included together with an optional opening 65. If an opening 65 is not present it is still preferable to have the dimple opening 62 for a tighter locking of the jaws 51 and 52. The jaws 51 and 52 rotate around both the inner hinge 63 and the hinge 60. Although the hinge is shown as a specific configuration, this is for illustrative purposes and any type of hinge device may be employed.

The sensor 113 can be removable or permanently attached to the present invention and is preferably placed on the second end 40 of the clamp 10. In this configuration it blends in with the locking mechanism 70, and is not obstructing other functionalities of the clamp 10. The preferred method of closing the present invention is a locking mechanism 69 that locks permanently, such as the first locking mechanism 70 together with an additional locking mechanism 72, both located substantially at the second end 40. The number and complexity of various locking mechanisms and features may vary as long as there is at least one locking mechanism 70 that achieves a permanent coupling.

Still referring to FIG. 1, the locking mechanism 69 is shown having a first locking mechanism 70 and an additional locking mechanism 72. The first locking mechanism 70 has a clasp 73, shown as a two opposite facing V shaped hooks 73b, and a latch 74, shown as two inwardly facing hooks 74a on either side of a gap 74c. To operate this closing mechanism 70, V shaped hooks 73a are forced into the gap 74c between the inwardly facing hooks 74a. The V shaped hooks 73a are strongly biased to take a V shape form. To get past the inwardly facing hooks 74a, the V shaped hooks 73a are forced together and then snap back into the shape of a V once permanently encased inside the recessed space 74b. The recessed space 74b does not afford any movement by V shaped hooks 73a, and the contour of the inwardly facing hooks 74a and of the V shaped hooks 73b is uniformly interlocking and snug, reinforcing the locking mechanism 69.

To further enhance the permanent nature of the locking mechanism 69, the opening 74c and the recessed space 74b may be enclosed by a side wall 15 (FIGS. 4 & 5) on both sides, which may or may not have additional inwardly facing hooks. A side wall 15 would completely block any access to the V shaped hooks 73b, once these are inside the recessed space 74b. If sidewalls have additional inwardly facing hooks 74a, the clasp 73 may have additional corresponding V shaped hooks 73a. These, together with the V shaped hooks 73a that are shown would then form an inverted tip of a spike, while the gap 74c with additional inwardly facing hooks 74a would form an opening with inwardly facing flaps.

The additional locking mechanism 72 has a hook 75 and a catch 76 and may provide a supplemental locking strength to the present invention. One skilled in the art will appreciate that other embodiments of permanent locking means are possible and may be used to enable to present invention.

The placement of the latch 74 and the clasp 73 is variable and either may be disposed on the first jaw 51 or on the second jaw 52. Once the clamp 10 is attached and locked around an umbilical cord or an umbilical stump of an infant, the serrated edge 50 makes it virtually impossible to remove it by either sliding the jaws 51 and 52 off the umbilical cord or stump or by opening up the locked mechanism 69, without having to cut the clamp 10. The serrated edge 50, although illustrated as serrated, may be other shapes. The edge may be any shape or functionality that achieves the desired purpose, such as, but not limited to, a flat surface, or an interlocking surface using any shape or pattern.

Still referring to FIG. 1, the sensor 113 may be located at the second end 40, as shown, or anywhere else throughout the clamp 10. The sensor 113, the first and second terminals 111 and 112, together with the wire 110 form the preferred integrated circuitry that enables the present invention to function as an active or passive security device. The wire 110 is discretely enclosed or embedded within the entire length of the first jaw 51, the second jaw 52, the hinge 60, the sensor 113, and within the first and second terminals 111 and 112. The sensor 113 contains a power source 120 (shown in later figs.) which may preferably be a battery, such as but not limited to a long life Lithium or Zink miniature battery, common in wrist watches and other small electronic devices. Alternatively, the power source 120 may be a rechargeable battery, such as but not limited to a nickel cadmium (NiCd), nickel metal hydride (NiMH), lithium ion (Li-ion), or lithium ion polymer (Li-ion polymer), in which case, the clamp 10 would include a charging terminal (not shown) that would connect to a charging device.

FIG. 2 discloses the clamp 10 shown in FIG. 1 in a closed position with the current now flowing along the wire 110, along the first and second jaws 51 and 52, the hinge 60, through the first terminal 111 and the second terminal 112, and into the sensor 113. Also shown in this figure are a first end 30, a second end 40, a clamp top 12, a clamp bottom 13, a serrated edge 50, a dimple opening 62, an opening 65, a clamp locking mechanism 69, having a first locking mechanism 70, an additional locking mechanism 72, a clasp 73, V shaped hooks 73b, a latch 74, inwardly facing hooks 74a, a gap 74b, a recessed opening 74c, a hook 75, a catch 76. The clamp locking mechanism is preferably disposable, but may be reuseable.

In an embodiment where the sensor 113 is a magnet, or rather contains an electromagnet, the current that flows through the wire 110 and into the sensor 113, would be wrapped around a segment or a plug of material capable of becoming a magnet. When the clamp 10 is closed, the magnetic field created by such a magnet causes a small amount of current to flow within the external detection device. This current flow within the external detection device is then detected as an intact clamp 10. If the clamp is cut during a security incident the sudden disappearance of this electrical current may trigger an alarm or other alerts. If a thief attempts to leave without removing the clamp 10, a set of sensor gates at entrances and exits will be able to detect a strong magnetic field passing within a close proximity, and trigger an alarm.

The sleeve 130 surrounds the first jaw 51 and the second jaw 52, as well as the inner hinge 63 and the first locking mechanism 70. It preferably affords resistance to tampering with the clamp 10. The sleeve 130 may be manufactured from any material, but preferably from a polymer, including but not limited to, plastic, silicone, or another hard, non-brittle polymer, or a vinyl polymer, such as PVC, or any other type of strong polymer. All materials used may be additionally tempered or reinforced. The sleeve 130 may also be manufactured out of a metal alloy that is non-conducting and non-oxidizing so as to avoid interference with various security devices and medical equipment, and to avoid exposing infants or others to by-products of oxidation, which may be toxic. A specific material used may additionally be non-magnetic and fire retardant. A material having memory may be utilized, so as to alert medical or security staff of any attempted tampering, for example, some plastics may begin to fragment into fibers that are difficult to remove or acquire scratches and other easily perceived signs of damage. The materials for the first and second jaws 51 and 52 may be any material, but should be preferably hypoallergenic, with plastic being the commonly used material.

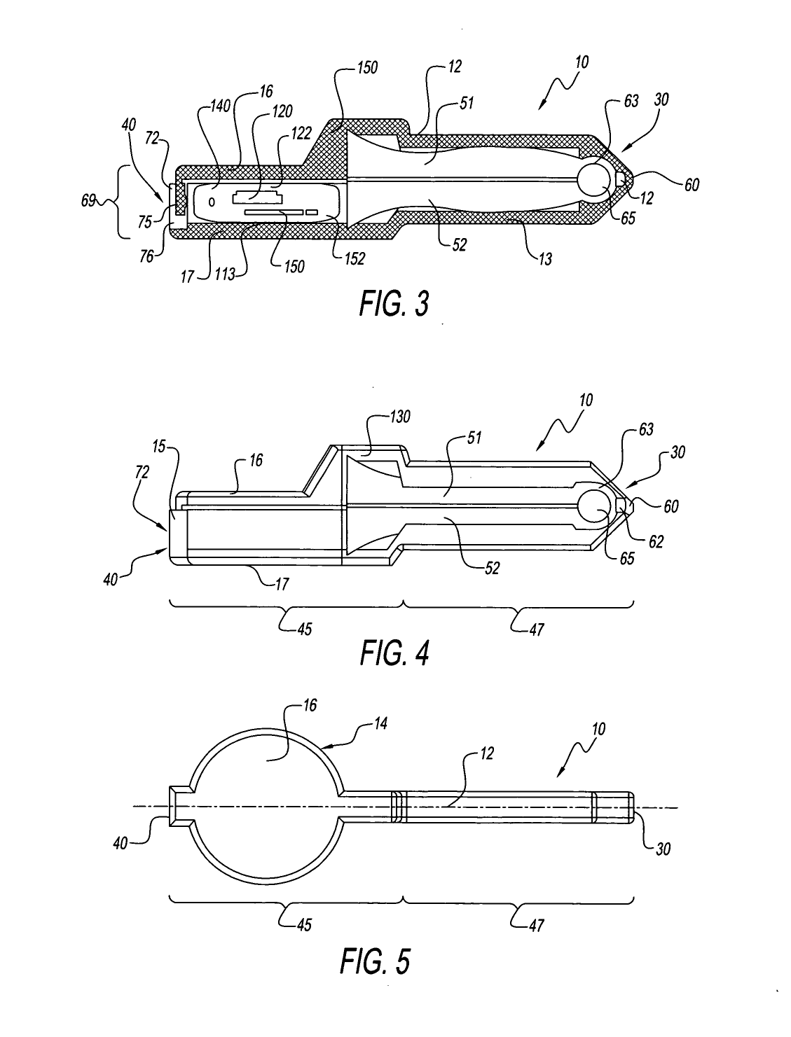

FIGS. 3-5 show an alternative embodiment of the present invention. Shown are a clamp 10, a clamp top 12, a clamp bottom 13, an enlarged section 14, sidewalls 15, a cover 16, a sensor dish 17, a first end 30, a second end 40, a large section 45, a small section 47, 45 & 47 look the same size a first jaw 51, a second jaw 52, a hinge 60, a dimple opening 62, an inner hinge 63, an opening 65, an additional locking mechanism 72, a hook 75, a catch 76, sensor 113, a power source 120, a circuit board 122, a sleeve 130, a receiver 140, a transmitter 150, and a memory chip 152. In this embodiment the first and second jaws 51 and 52 form the small section 47, whereas the cover 16 and dish 17 that contain and cover the sensor 113 form the large section 45. Note that the clamp top 12 is shaped in a way that is convenient for pressing by a human finger. The dimensions of the small section 47 for alternative and preferred embodiments are preferably between 0.3 and 0.5 of an inch in height and between 0.24 and 0.34 of an inch in width. The dimensions of the large section 45 for alternative and preferred embodiments may be between 0.6 and 0.91 of an inch high with the diameter of the enlarge section between 0.5 and 1 inch in width. The overall length of the clamp 10 may be between 2 and 4 inches. The appearance of the clamp 10 or of the large and small sections 45 and 47 may be varied. The clamp 10, may be substantially straight without any pronounced curves or sectional bending.

In FIGS. 3-5, only the additional locking mechanism 72 is shown, (the clamp locking mechanism 70 is not shown) and may serve as the primary locking mechanism 69 for this particular embodiment. Alternatively a first locking mechanism 70 may have been used instead. Additionally, the locking mechanism 69 may be enhanced by a magnetic locking mechanism that would attract the cover 16 to the sensor dish 17. This magnet may be activated when the clamp 10 is closed, with the current flowing through the closed circuit via the wire 110 and terminals 111 and 112 (not shown in this figure). In this embodiment the sidewall 15 surrounds the dish 17 and either conceals the additional locking mechanism 72 entirely, or forms a catch 76.

The sensor 113 may transmit and/or receive a variety of signal types such as, but not limited to radio frequency identification (RFID), ultrasound identification (USID), wireless network signals or (Wi-Fi), microwaves, or an electric current from a strong magnetic field. The type of signal transmitted or received directly impacts the type of an external detection device that may be used with the present invention. In the present invention, the clamp 10 may begin emitting signals or other types of electromagnetic radiation, as soon as the clamp 10 closes and the first terminal 111 and the second terminal 112 are in physical connection with each other. Thus the closing of the locking mechanism 69 enables the direct current from the power source to flow through the wire 110 and provide power to the other components of the sensor 113.

In an embodiment where the sensor 113 is an RFID device, the external detection device will need to be able to transmit, receive and interpret radio signals. The sensor 113 would typically have an internal memory chip 152, that can be written to once or many times. The memory chip 152 may be the same no matter what type of electromagnetic radiation is being processed by the sensor 113. The external detection device would obtain all information contained on this internal memory chip 152, once the sensor 113 and the external detection device have an established communication. This information may include, but is not limited to, a unique identifier of the particular clamp 110. In security records of the infant care facility this unique identifier will likely associated with a particular infant carrying this particular clamp 10. Additionally the sensor 113 may be able to detect an infant's body temperature and communicate this information to the external detection device 170. This may be important in helping to prevent SIDS. For example, a sudden drop or rise of body temperature, or a drop or rise of temperature below or above a certain point, may be a sign of distress, which can be acted on by the medical staff. The medical staff may be alerted to this distress by the sensor 113.

An RFID enabled sensor 113, may have a power source 120 that may be a battery, a receiver 140 for receiving polling and probing signals from an external detection device, a transmitter 150 to respond to such polling or probing requests, and a memory chip 152. An RFID enabled sensor 113 may also be powered by the probing or polling signal from the external detection device. The receiver 140 may contain an integrated antenna 114 (FIG. 7) for radio signals, or the antenna 114 may be stand alone. A poling or probing request, or an active signal from the sensor 113, would update the external detection device 170 with all identification information stored on a memory chip 152. An RFID sensor 113 would preferably be small notwithstanding the size of the battery, receiver, antenna 114 or other components of the present invention. The receiver 140 on the sensor 113 or an equivalent receiver on the external detection device 170 may be paired up with a transmitter 150 or an equivalent transmitter on the external detection device, forming a transponder. U.S. Pat. No. 4,384,288 is referenced in the present application for a more comprehensive overview of an RFID technology. An RFID enabled sensor 113 may be located even when the clamp 10 and the internal wire 110 has been severed or damaged, since the power necessary to drive the transmitter 150 would be supplied by the signal from the external detection device 170.

In an embodiment where the sensor 113 is a USID, the external detection device 170 would have a plurality of microphones that would detect and locate the high frequency polling sounds emitted by the sensor 113. The sensor 113 would then utilize a specialized speaker element (not shown) coupled to a power source 120 to generate ultrasonic ping messages at predetermined intervals. In this embodiment an alarm is triggered when an expected polling sound from the sensor 113 is not detected by the external detection device 170. This would indicate the device has been cut or damaged, or that the infant has been removed from the monitored area. A microwave enabled sensor would operate in a similar fashion with the difference being the frequency of wavelength being transmitted or received by the present invention.

An embodiment of a sensor 113 that would connect to a wireless computer network may send out status and informational signals much the same way as in an ultrasound embodiment described above. However, in the Wi-Fi embodiment, the external detection device 170 may likely be a wireless antenna 114 that may be connected to an access point on a computer network that links monitoring equipment to the plurality of clamps 10 that are attached to the infants at the monitored facility. Cutting, damaging or carrying away of clamp 10 would trigger an alarm since the system would fail to receive a timely “I am alive” message at the next expected interval. Additionally, such a wireless antenna 114 may be calibrated to detect an especially strong signal, such as when an infant is being carried through a set of exit or entry gates (not shown) equipped with such an antenna 114. The strong signal from the claimed invention would be especially strong, when in close proximity with such a gate and would therefore trigger an alarm.

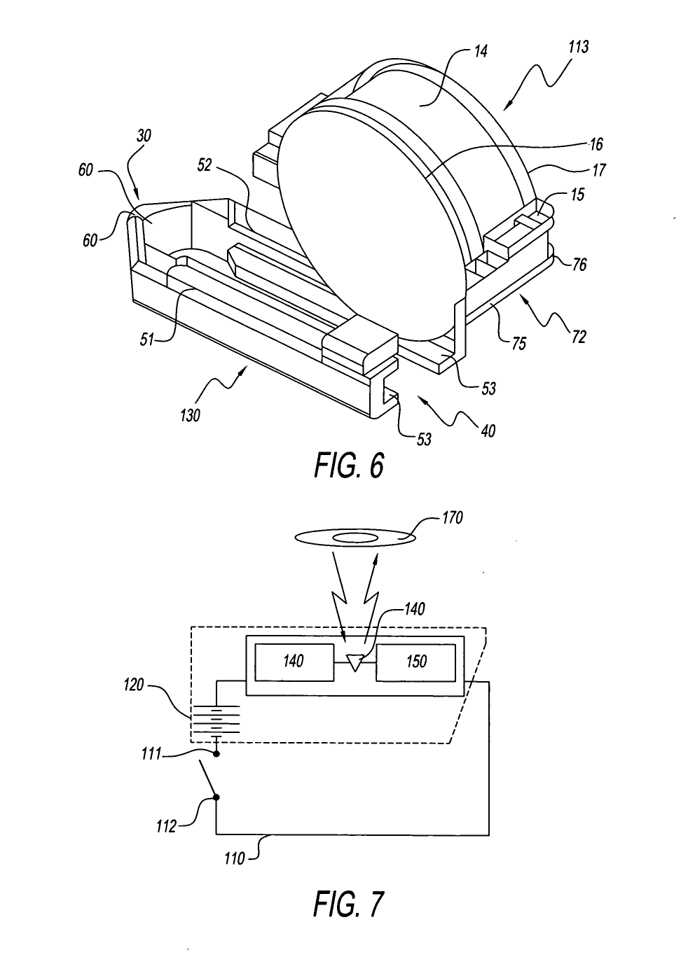

FIG. 6 shows another alternative embodiment of the present invention. Shown is an enlarged section 14, sidewall 15, a cover 16, a dish 17, a first end 30, a second end 40, a first jaw 51, a second jaw 52, a channel 53, a hinge 60, a dimple opening 62, an additional locking mechanism 72, a hook 75, a catch 76, sensor 113, and a sleeve 130. In this embodiment a conventional umbilical cord clamp may be slipped into the channels 53 of the sleeve 130. The cover 16, the dish 17 and the sensor 113 have been moved to the side of the sleeve, to enable easy insertion of a conventional clamp into the channel 53. However, the cover 16 and the dish 17 may also be disposed at the second end 40 as in the prior figs. The same signaling technology may be employed in this embodiment as described above.

FIG. 7 is a schematic representation of the circuitry within the present invention. Shown is a sensor 113, represented by a dashed line and the components making up the sensor 113. Also shown are the power source 120; a switch that is formed between the first terminal 111 and the second terminal 112; a wire 110 that is embedded in within the first and second jaws 51 and 52 and within the hinge 60; a receiver 140 and a transmitter 150, both connecting to an antenna 114. The antenna 114 is shown sending and receiving signals with an external detection device 170.

The present invention may be placed around an umbilical cord or an umbilical cord stump of an infant, and carry on all medically necessary functionality of a conventional clamp. Once the present invention is closed, it is permanently locked. The sensor 113 establishes communication with an external detection device 170, such as an external antenna, access point or a plurality of these or other types of sensors. Additionally, a transmitter 150 within the sensor 113 of sufficient strength may be able to communicate with a satellite receiver. These types of sensors are well known in the art. There may also likely be some kind of interaction with a tracking technology, most likely a computer running a version of tracking software. The tracking technology may have a connection to the hospital's alarm or external speaker system or another type of warning or an alert implementation, which is triggered when a security incident is detected.

It is preferable that the jaws 51 and 52 may be between 1 and 2 inches long. It is also preferable that the total length of the supervision device embodied by the present invention may be between 1 and 4 inches long.

Although this invention has been described with a certain degree of particularity, it is to be understood that the present disclosure has been made only by way of illustration and that numerous changes in the details of construction and arrangement of parts may be resorted to without departing from the spirit and the scope of the invention.

I claim:

1. An infant supervision device comprising;
a clamp, having a first end and a second end, and a first jaw and a second jaw,
wherein said first and second jaws rotate around a hinge disposed on said first end;
a permanently locking mechanism disposed on said second end;
a wire emanating from said first end and said second end, and wherein said wire is discretely enclosed within an entire length of said first jaw and said second jaw and said hinge, and wherein said wire forms a circuit when said permanently locking mechanism is closed;
a sensor permanently disposed on said clamp, wherein said sensor is capable of communicating with an external detection device; and
a power source, wherein said power source powers said sensor and said circuit.

2. The infant supervision device of claim 1, wherein said clamp is an umbilical cord clamp.

3. The infant supervision device of claim 1, wherein said sensor is a magnet.

4. The infant supervision device of claim 1, wherein said external reader and said sensor communicate using a protocol selected from a group comprising radio waves, microwaves, or a wireless network protocol.

5. The infant supervision device of claim 1, wherein said power source is a battery.

6. The infant supervision device of claim 1, wherein said jaws are between 1 and 2 inches long.

7. The infant supervision device of claim 1, wherein said clamp is made of a non-conducting metallic alloy.

8. The infant supervision device of claim 4, wherein said receiver and said transmitter is capable of serving as a transponder for said supervision device.

9. The infant supervision device of claim 1, wherein said sensor is uniquely identified by said external detection device.

10. The infant supervision device of claim 1, wherein said sensor is capable of alerting said external detection device of a structural breach of said clamp.

11. The infant supervision device of claim 13, wherein said structural breach disables said sensor.

12. The infant supervision device of claim 1, wherein the total length of said supervision device is between 1 and 4 inches.

13. The infant supervision device of claim 1, wherein said sensor is disposed on said second end of said clamp.

14. The infant supervision device of claim 1, wherein said power source is rechargeable.

15. The infant supervision device of claim 1, further comprising a sleeve capable of permanently encasing said clamp.

16. The infant supervision device of claim 1, wherein said sensor is capable of sending tracking information to said external reader.

17. The infant supervision device of claim 4, wherein said receiver or said transmitter are capable of communicating via satellite to said external reader.

18. The infant supervision device of claim 1, wherein said sensor is capable of detecting infant's body temperature.

19. The infant supervision device of claim 1, wherein the permanently locking mechanism is further comprised of a first locking mechanism and an additional locking mechanism.

20. The infant supervision device of claim 1, wherein the permanently locking mechanism is further comprised of a first locking mechanism or an additional locking mechanism and a magnetic locking mechanism, wherein such magnetic locking mechanism is powered by said power source.
